(19) United States
(12) Patent Application Publication
(10) Pub. No.: US 2025/0275954 A1
(43) Pub. Date: Sep. 4, 2025

(54) USE OF KYNURENIC ACID RESISTING HYPERURICEMIA AND STRUCTURAL ANALOG THEREOF IN PREPARATION OF XOD INHIBITOR

(71) Applicant: JIMEI UNIVERSITY, Xiamen (CN)

(72) Inventors: Jian LI, Xiamen (CN); Lingyu ZHANG, Xiamen (CN); Daren WU, Xiamen (CN)

(21) Appl. No.: 19/211,138

(22) Filed: May 16, 2025

Related U.S. Application Data
(63) Continuation of application No. PCT/CN2024/096975, filed on Jun. 3, 2024.

(30) Foreign Application Priority Data
Nov. 20, 2023 (CN) 202311550882.4

(51) Int. Cl. A61K 31/47; A61P 19/06

(52) U.S. Cl. CPC A61K 31/47; A61P 19/06

(57) ABSTRACT

The present disclosure relates to use of kynurenic acid (KA) resisting hyperuricemia (HUA) and a structural analog thereof in the preparation of xanthine oxidase (XOD) inhibitors, wherein the KA and the structural analog have good inhibitory activity on XOD; the KA has no significant effect on a growth status and a body weight of mice, and oral administration of low, medium or high doses of the KA can significantly reduce serum creatinine and blood urea nitrogen levels in mice, respectively, and effectively inhibit hepatic XOD enzyme activity in mice and thus reduce hepatic uric acid levels in a concentration-dependent manner; low, medium or high doses of KA can significantly reduce serum ADA enzyme activity in hyperuricemic mice, limiting the production of uric acid precursors, and the KA can be used as a medicament for ameliorating or treating HUA.

### CROSS-REFERENCE TO RELATED APPLICATIONS

This application is a continuation of International Patent Application No. PCT/CN2024/096975, filed on Jun. 3, 2024, which claims the benefit of priority from Chinese Patent Application No. 202311550882.4, filed on Nov. 20, 2023. The content of the aforementioned applications, including any intervening amendments thereto, are incorporated herein by reference.

### TECHNICAL FIELD

The present disclosure relates to the technical field of medicine, in particular to use of kynurenic acid resisting hyperuricemia and a structural analogue thereof in the preparation of xanthine oxidase (XOD) inhibitors.

### BACKGROUND

Uric acid (UA) is a final product formed after metabolism of purines in the liver, muscle and intestinal tract, and is mainly formed after decomposition of adenylic acid and guanine. Under normal circumstances, the production and excretion of UA in the human body basically maintain a dynamic balance, with 70% being excreted from the kidneys and 30% being excreted through the intestinal tract. Hyperuricemia (HUA) is a metabolic disease in which excessive uric acid production and/or reduced UA excretion are caused by abnormal purine metabolism, causing blood uric acid to exceed a normal range. Due to the absence of urate oxidase in the human body, UA is produced as an end product of hypoxanthine catabolismby xanthine oxidase (XOD). XOD, which promotes the synthesis of uric acid, is predominantly expressed in the liver and intestinal tract.

In recent years, with the rapid development of economics and lifestyle changes, the incidence of HUA worldwide has increased significantly and is in a younger trend. According to recent epidemiological data, the prevalence of HUA is approximately 11.9% in the United States, while in China, the rate has increased to over 13%, particularly in urban areas. These statistics underscore the growing burden of HUA on public health systems worldwide. Not only is HUA a significant risk factor for gout, but it is also closely linked to various diseases such as kidney disease, hypertension, diabetes, cardiovascular disease, and similar conditions. Prompt and effective administration of UA lowering therapy is crucial to reducing urate deposition in the body, decreasing the risk of gout occurrence, alleviating kidney damage, and reducing the occurrence of other complications.

Relevant therapeutic strategies for HUA include the use of XOD inhibitors such as allopurinol, a purine structural analog that selectively inhibits XOD activity to reduce UA production. Despite its efficacy, long-term allopurinol use is associated with adverse effects, including allergic reactions, rash, and hepatorenal toxicity. Given these limitations, there is an urgent need for safe, effective, and affordable therapeutic agents or functional foods to alleviate the adverse effects of HUA. Recent advancements have explored the potential of natural products as XOD inhibitors with minimal side effects. In particular, kynurenic acid and its structural analogs have shown promising XOD inhibitory activity, representing a novel approach for HUA management.

### SUMMARY

The present disclosure aims to solve one of the technical problems in the related art at least to some extent. To this end, an object of the present disclosure is to provide use of KA and a structural analog thereof in the preparation of XOD. KA has the following structural formula:

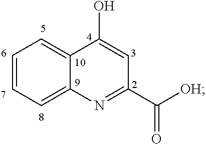 [SMILES: O=C(O)c1cc(O)c2ccccc2n1]

the structural analog of the KA having the following general structural formula:

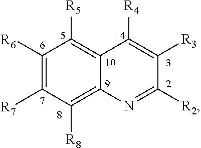

wherein R₃ is ethyl carboxylate, R₄ is hydroxy, and R₆ is methoxy; or R₃ is carboxyethyl ester, R₄ is hydroxy, and R₈ is methoxy; or R₃ is ethyl carboxylate, and R₄ is hydroxy; or R₃ is carboxylic acid and R₄ is hydroxy; or R₃ is carboxylic acid, and R₆ is hydroxy; or R₄ is hydroxy; or R₂ is ethyl carboxylate, and R₄ is hydroxy; or R₂ is carboxylic acid, R₄ is hydroxy, and R₈ is hydroxy; or R₂ is carboxylic acid, and R₅ is hydroxy; or R₂ is hydroxy, and R₄ is carboxylic acid; or R₂ is carboxylic acid; or R₂ is carboxylic acid, and R₃ is hydroxy; or R₂ is carboxylic acid, and R₈ is hydroxy; or R₆ is carboxylic acid and R₈ is hydroxy; or R₇ is carboxylic acid and R₈ is hydroxy; or R₄ is hydroxy and R₇ is carboxylic acid.

Optionally, the structural analog of the KA is ethyl 4-hydroxy-6-methoxyquinoline-3-carboxylate, ethyl 4-hydroxy-8-methoxyquinoline-3-carboxylate, ethyl 4-hydroxyquinoline-3-carboxylate, 4-hydroxyquinoline-3-carboxylic acid, 6-hydroxyquinoline-3-carboxylic acid, 4-hydroxyquinoline, ethyl 4-hydroxyquinoline-2-carboxylate, xanthurenic acid, 5-hydroxyquinoline-2-carboxylic acid, 2-hydroxyquinoline-4-carboxylic acid, quinoline-2-carboxylic acid, 3-hydroxyquinoline-2-carboxylic acid, 8-hydroxyquinoline-2-carboxylic acid, 8-hydroxyquinoline-6-carboxylic acid, 8-hydroxyquinoline-7-carboxylic acid, or 4-hydroxyquinoline-7-carboxylic acid.

According to the use in an example of the present disclosure, the results of an in vitro XOD inhibitory activity assay show that the in vitro XOD inhibitory activity of 6-Hydroxyquinoline-3-carboxylic acid is optimal, with an IC₅₀ of 0.50±0.13 mM. 3-Hydroxyquinoline-2-carboxylic acid, 2-Hydroxyquinoline-4-carboxylic acid and KA all show good XOD inhibitory activity, with IC₅₀ of 0.97±0.05 mM, 1.61±0.07 mM and 1.99±0.26 mM, respectively.

In a second aspect of the present disclosure, an example of the present disclosure provides use of KA in the preparation of a medicament for ameliorating or treating HUA.

According to the use in the example of the present disclosure, ingestion of the KA is tested to have no significant effect on a growth status and a body weight of mice. Oral administration of low (7 mM/kg/day), medium (35 mM/kg/day) or high (70 mM/kg/day) doses of the KA can significantly reduce serum creatinine (CRE) and blood urea nitrogen (BUN) levels in mice, respectively, and effectively inhibit hepatic XOD enzyme activity and thus reduce hepatic UA levels in a concentration-dependent manner. In experimental groups, low, medium or high doses of KA can effectively reduce serum ADA enzyme activity in hyperuricemic mice, limiting the production of uric acid precursors.

In a third aspect of the present disclosure, an example of the present disclosure provides a medicament for ameliorating or treating hyperuricemia, including kynurenic acid and a pharmaceutically acceptable carrier.

According to the medicament in the example of the present disclosure, the ingestion of KA has been tested and found to have no significant impact on a growth status or body weight of mice. Oral administration of low (7 mM/kg/day), medium (35 mM/kg/day) or high (70 mM/kg/day) doses of KA significantly reduces serum creatinine (CRE) and blood urea nitrogen (BUN) levels in mice, respectively, and effectively inhibit hepatic XOD enzyme activity and thus reduce hepatic uric acid levels in a concentration-dependent manner. In experimental groups, low, medium or high doses of KA can effectively reduce serum ADA enzyme activity in hyperuricemic mice, limiting the production of uric acid precursors.

In a fourth aspect of the present disclosure, an example of the present disclosure provides a medicament for ameliorating or treating KA, including a KA structural analog and a pharmaceutically acceptable carrier, the KA structural analog being ethyl 4-hydroxy-6-methoxyquinoline-3-carboxylate, ethyl 4-hydroxy-8-methoxyquinoline-3-carboxylate, ethyl 4-hydroxyquinoline-3-carboxylate, 4-hydroxyquinoline-3-carboxylic acid, 6-hydroxyquinoline-3-carboxylic acid, 4-hydroxyquinoline, ethyl 4-hydroxyquinoline-2-carboxylate, xanthurenic acid, 5-hydroxyquinoline-2-carboxylic acid, 2-hydroxyquinoline-4-carboxylic acid, quinoline-2-carboxylic acid, 3-hydroxyquinoline-2-carboxylic acid, 8-hydroxyquinoline-2-carboxylic acid, 8-hydroxyquinoline-6-carboxylic acid, 8-hydroxyquinoline-7-carboxylic acid, or 4-hydroxyquinoline-7-carboxylic acid.

According to the medicament in the example of the present disclosure, the results of an in vitro XOD inhibitory activity assay show that the in vitro XOD activity of 6-Hydroxyquinoline-3-carboxylic acid is optimal, with an IC₅₀ of 0.50±0.13 mM. 3-Hydroxyquinoline-2-carboxylic acid, 2-Hydroxyquinoline-4-carboxylic acid and KA all show good XOD inhibitory activity, with IC₅₀ of 0.97±0.05 mM, 1.61±0.07 mM and 1.99±0.26 mM, respectively. Thus, the KA structural analog can be used as a medicament for ameliorating or treating HUA.

The additional aspects and advantages of the present disclosure will be partially outlined in the following description, while others will be evident from this description or can be discerned through the application of the present disclosure.

### BRIEF DESCRIPTION OF THE DRAWINGS

FIG. 1 shows the chemical structure of KA according to an example of the present disclosure;

FIG. 2 shows changes in body weight of mice at different times according to an example of the present disclosure;

FIG. 3 shows changes in serum uric acid levels in mice according to an example of the present disclosure;

FIG. 4 shows changes in serum XOD enzyme activity in mice according to an example of the present disclosure;

FIG. 5 shows changes in serum creatinine (CRE) levels in mice according to an example of the present disclosure;

FIG. 6 shows changes in blood urea nitrogen (BUN) levels in mice according to an example of the present disclosure;

FIG. 7 shows changes in hepatic XOD enzyme activity in mice according to an example of the present disclosure;

FIG. 8 shows changes in hepatic uric acid levels in mice according to an example of the present disclosure;

FIG. 9 shows changes in serum adenosine deaminase activity (ADA) in mice according to an example of the present disclosure;

FIG. 10A to 10R shows xanthine oxidase inhibitory activity of quinoline structural analogs at various concentrations and allopurinol according to an example of the present disclosure;

FIG. 11 shows XOD inhibitory activity of kynurenic acid (KA) and structural analogs thereof at a concentration of 1 mM according to an example of the present disclosure.

### DETAILED DESCRIPTION

The technical solutions of the present disclosure are illustrated below by specific examples. It should be understood that one or more method steps mentioned in the present disclosure do not exclude the presence of other method steps before and after the combined steps or the possibility of inserting other method steps between those steps explicitly mentioned; and it should also be understood that these examples are merely illustrative of the present disclosure and are not intended to limit the scope of the present disclosure. Moreover, unless otherwise specified, the numbering of the method steps is merely a convenient tool for identifying the method steps, and is not intended to limit the order in which the method steps are arranged or to limit the scope of implementation of the present disclosure, and changes or adjustments of their relative relationships, without substantially changing the technical content, shall also be regarded as being within the scope of implementation of the present disclosure.

In order to better understand the above technical solutions, the examples of the present disclosure are described in more detail below. Although the examples of the present disclosure have been illustrated, it should be understood that the present disclosure may be implemented in various forms and should not be limited by the examples set forth herein. Rather, these examples are provided so that the present disclosure will be more thoroughly understood, and the scope of the present disclosure may be fully conveyed to those skilled in the art.

The test materials used in the present disclosure are all common commercial products and are commercially purchased.

KA structural analogs may be as follows:
(1) Ethyl 4-hydroxy-6-methoxyquinoline-3-carboxylate
wherein: R₃ is ethyl carboxylate, R₄ is hydroxy, and R₆ is methoxy

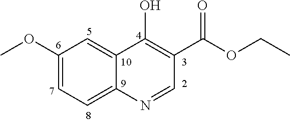 [SMILES: CCOC(=O)c1cnc2ccc(OC)cc2c1O]

(2) Ethyl 4-hydroxy-8-methoxyquinoline-3-carboxylate
wherein: R₃ is carboxyethyl ester, R₄ is hydroxy, and R₈ is methoxy

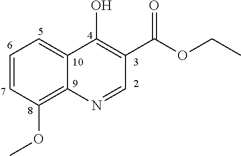 [SMILES: CCOC(=O)c1cnc2c(OC)cccc2c1O]

(3) Ethyl 4-Hydroxyquinoline-3-carboxylate
wherein: R₃ is ethyl carboxylate, and R₄ is hydroxy

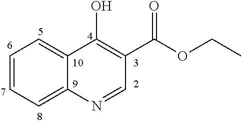 [SMILES: CCOC(=O)c1cnc2ccccc2c1O]

(4) 4-Hydroxyquinoline-3-carboxylic acid
wherein: R₃ is carboxylic acid, and R₄ is hydroxy

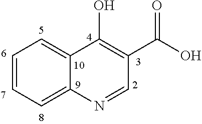 [SMILES: O=C(O)c1cnc2ccccc2c1O]

(5) 6-Hydroxyquinoline-3-carboxylic acid
wherein: R₃ is carboxylic acid, and R₆ is hydroxy

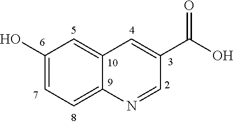 [SMILES: O=C(O)c1cnc2ccc(O)cc2c1]

(6) 4-Hydroxyquinoline
wherein: R₄ is hydroxy

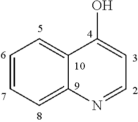 [SMILES: Oc1ccnc2ccccc12]

(7) Ethyl 4-hydroxyquinoline-2-carboxylate
wherein: R₂ is ethyl carboxylate, and R₄ is hydroxy

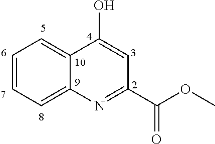

(8) Xanthurenic acid
wherein: R₂ is carboxylic acid, R₄ is hydroxy, and R₈ is hydroxy

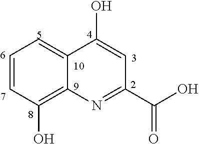 [SMILES: O=C(O)c1cc(O)c2cccc(O)c2n1]

(9) 5-Hydroxyquinoline-2-carboxylic acid
wherein: R₂ is carboxylic acid, and R₅ is hydroxy

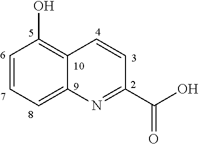 [SMILES: O=C(O)c1ccc2c(O)cccc2n1]

(10) 2-Hydroxyquinoline-4-carboxylic acid
wherein: R₂ is hydroxy, and R₄ is carboxylic acid

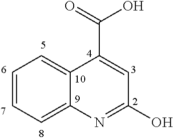 [SMILES: O=C(O)c1cc(O)nc2ccccc12]

(11) Quinoline-2-carboxylic acid
wherein: R₂ is carboxylic acid

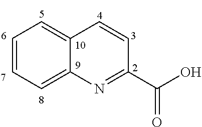 [SMILES: O=C(O)c1ccc2ccccc2n1]

(12) 3-Hydroxyquinoline-2-carboxylic acid
wherein: R₂ is carboxylic acid, and R₃ is hydroxy

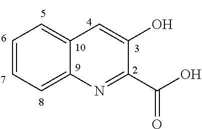 [SMILES: O=C(O)c1nc2ccccc2cc1O]

(13) 8-Hydroxyquinoline-2-carboxylic acid
wherein: R₂ is carboxylic acid, and R₈ is hydroxy

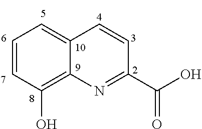 [SMILES: O=C(O)c1ccc2cccc(O)c2n1]

(14) 8-Hydroxyquinoline-6-carboxylic acid
wherein: R₆ is carboxylic acid, and R₈ is hydroxy

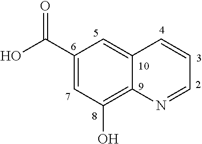 [SMILES: O=C(O)c1cc(O)c2ncccc2c1]

(15) 8-Hydroxyquinoline-7-carboxylic acid
wherein: R₇ is carboxylic acid, and R₈ is hydroxy

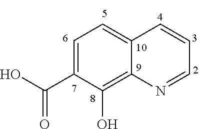 [SMILES: O=C(O)c1ccc2cccnc2c1O]

(16) 4-Hydroxyquinoline-7-carboxylic acid
wherein: R₄ is hydroxy, and R₇ is carboxylic acid

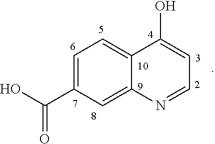 [SMILES: O=C(O)c1ccc2c(O)ccnc2c1]

The present disclosure will be described below with reference to specific examples, and it should be noted that these examples are merely descriptive and do not limit the present disclosure in any way.

### Example 1: Effect of Kynurenic Acid On Body Weight of Hyperuricemic Mice

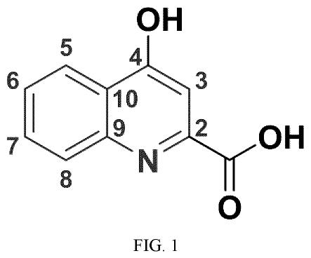

Kynurenic acid (KA), a chemical structure of which is shown in FIG. 1, was sourced from Sigma-Aldrich (Shanghai) Trading Co., Ltd.

SPF-grade male Kunming mice (8 weeks old), 35±5 g, were purchased from Changzhou Cavens Laboratory Animal Co., Ltd. Animal quality was tested by Suzhou Xishan Biotechnology Co., Ltd.

Experimental mice were housed in a clean animal feeding room, and feeding and experimental operations were performed according to the standards of the Animal Experimental Center, and the animal feeding room and an operating workbench were cleaned and disinfected daily. All experimental mice were housed in a comfortable environment (temperature: 25±2° C., humidity: 40%-70%, 12 hours of day and night cycle) with good ventilation and daily supplementation of feed and drinking water.

36 SPF-grade male Kunming mice with relatively consistent posture and good health were randomly divided into a normal control group, a model group, a positive control group, a kynurenic acid low dose group (KA-L), a kynurenic acid medium dose group (KA-M) and a kynurenic acid high dose group (KA-H), with six experimental mice in each group. The normal control group received daily gavage of an equal amount of CMC-Na solution and injection of an equal amount of saline, and the remaining groups received daily gavage of 500 mg/kg hypoxanthine (HX) and intraperitoneal injection of 300 mg/kg potassium oxazinate (PO). The positive control group received daily gavage of 35 mM/kg allopurinol, and the other experimental groups received daily gavage of 7 mM/kg KA (KA-L), 35 mM/kg KA (KA-M), and 70 mM/kg KA (KA-H), respectively. Oral gavage was continued for 21 days. Data were analyzed for correlation and significance by using SPSS 23.0 software, and differences between the groups were compared by using one-way ANOVA, *p<0.05 indicates a significant difference, **p<0.01 indicates a very significant difference, and the data were expressed as X̅±SD.

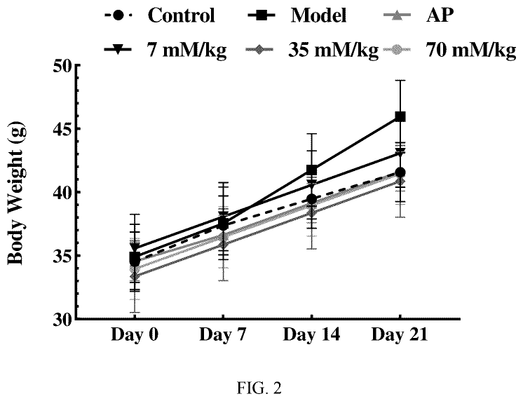

The change in body weight of the mice in each group at different time points was measured after 21 days of feeding experiment, and the results are shown in FIG. 2. There were no significant differences in the body weight of the mice in each group on day 7 of the intervention, and the body weight of the mice in the model group increased compared with other groups on day 21 of the intervention, but there were no significant differences (p>0.05) compared with the body weight of the mice in the other groups. The results showed that gavage of KA to the mice did not affect their growth status and body weight.

### Example 2: Effect of Kynurenic Acid on Serum Uric Acid in Hyperuricemic Mice

At the end of the experiment on day 21, the mice in each group of Example 1 were subjected to blood collection from eyeballs of living bodies after being fasted for solids and liquids for 12 h. After sufficient blood samples were collected, they were allowed to stand at room temperature for 30 min and then centrifuged (3500 rpm, 10 min). A supernatant was taken and stored at −20° C. for standby application.

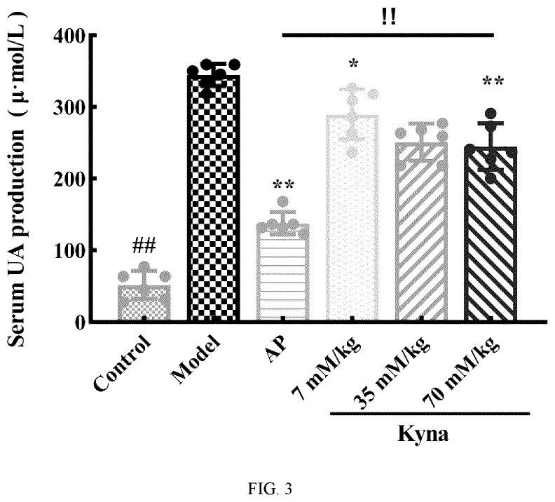

Serum uric acid levels in target mice were detected by using a uric acid (UA) test kit. The serum uric acid level is an important evaluation index of drug improvement in hyperuricemia. As shown in FIG. 3, the serum uric acid level in the mice of the blank control group was significantly lower than that of the model group (p<0.01), and the positive control group (allopurinol) significantly inhibited the increase in mouse serum uric acid level (p<0.01). All three dose groups of KA could significantly reduce serum uric acid levels in the mice (p<0.01), with the inhibitory effect of KA-H on the serum uric acid levels in the mice being most pronounced.

### Example 3: Effect of Kynurenic Acid on Serum XOD Enzyme Activity in Hyperuricemic Mice

Serum samples of Example 2 were taken, and the XOD enzyme activity levels in the mouse serum samples were determined by using a xanthine oxidase (XOD) assay kit (a colorimetric method), and experimental operations and data calculation were performed in strict accordance with the kit instructions.

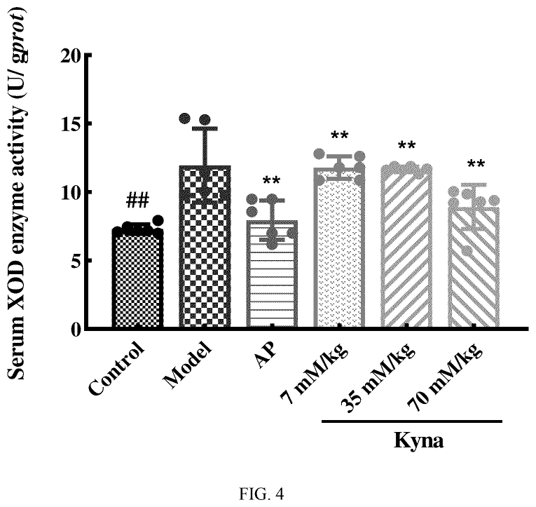

As shown in FIG. 4, the XOD enzyme activity levels in the mice of the model group remained at a higher level compared with the blank control group. The positive control group could significantly reduce serum XOD enzyme activity levels in hyperuricemic mice (p<0.01), and KA-L, KA-M and KA-H could all reduce serum XOD enzyme activity in hyperuricemic mice to varying degrees (p<0.01), which was positively correlated with the concentration of KA.

### Example 4: Effect of Kynurenic Acid on Serum Creatinine and Blood Urea Nitrogen in Hyperuricemic Mice

Serum samples of Example 2 were taken and the CRE content of the mouse serum samples was measured by using a creatinine (CRE) assay kit (sarcosine oxidation), and experimental operations and data calculation were performed in strict accordance with the kit instructions.

Serum samples of Example 2 were taken and the blood urea nitrogen (BUN) content in the mouse serum samples was measured by using a blood urea nitrogen (BUN) assay kit (a urease method), and experimental operations and data calculation were performed in strict accordance with the kit instructions.

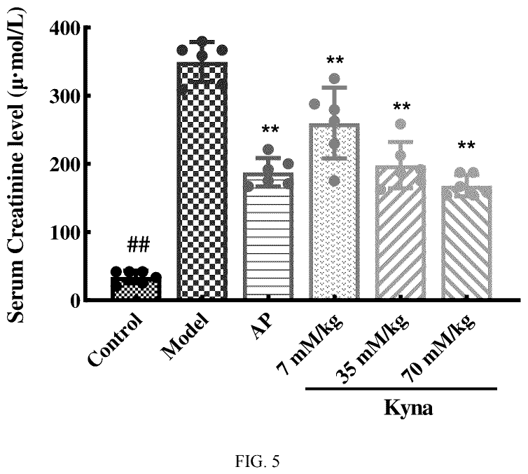

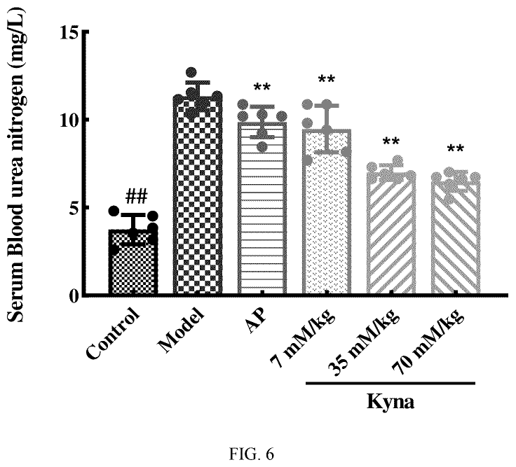

Creatinine (CRE) and blood urea nitrogen (BUN) are important indicators reflecting the kidney function. As shown in FIGS. 5 and 6, serum CRE and BUN of the mice in the model group were significantly increased (p<0.01) compared with the blank control group, indicating that administration of potassium oxazinate (PO) and hypoxanthine (HX) to the mice resulted in impairment of the kidney function in the mice. KA could significantly decrease serum CRE and BUN in the mice compared with the model group (p<0.01) and showed better concentration dependence. Also, KA-M and KA-H could reduce BUN levels in the mice more significantly compared with the positive control group (p<0.01). It was indicated that KA could improve renal dysfunction in the mice by reducing serum CRE and BUN in the hyperuricemic mice.

### Example 5: Effect of Kynurenic Acid on Hepatic Uric Acid and Xanthine Oxidase (XOD) in Hyperuricemic Mice

On day 21, the mice in each group of Example 1 were quickly sacrificed by cervical dislocation after being fasted for solids and liquids for 12 h and livers were collected for weighing and subpackaging. 100 mg of mouse liver samples were triturated in normal saline, and centrifuged at 3500 rpm at 4° C. for 15 min, and a supernatant homogenate portion was taken and placed at −20° C. for standby application. Uric acid levels in the mouse liver homogenate samples were determined by using the method described in Example 2. XOD enzyme activity levels in the mouse liver homogenate samples were determined by using the method described in Example 3.

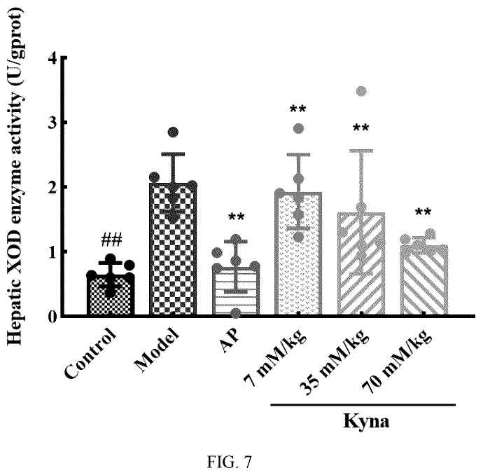

The hepatic XOD enzyme activity in the mice was determined and the results are shown in FIG. 7. The hepatic XOD enzyme activity levels were significantly increased in the model group compared with the blank control group and the positive control group (p<0.01). Furthermore, the hepatic XOD enzyme activity levels in the mice were decreased significantly (p<0.01) after administration of KA to the mice and correlated positively with the concentration of KA.

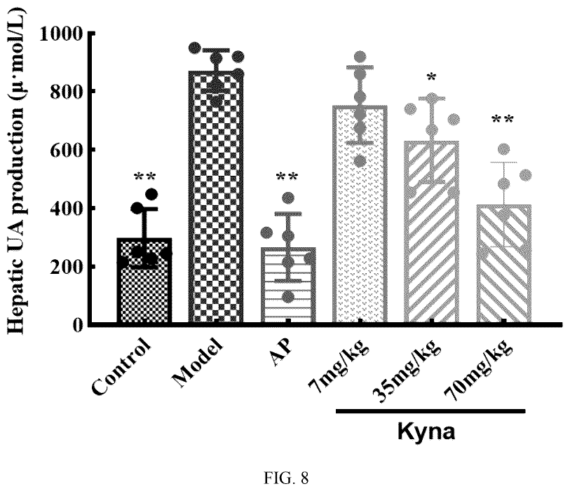

The uric acid content in the livers of the mice were determined and the results are shown in FIG. 8. The uric acid content in the livers of the mice could be significantly reduced in the positive control group compared with the blank control group (p<0.01), and reached a level comparable to that in the blank control group. After administration of different concentrations of KA, the uric acid content in the livers of the mice showed a gradient decline (p<0.01). The above results indicate that KA can effectively inhibit hepatic XOD enzyme activity in the mice, thereby reducing hepatic uric acid levels.

Example 6: Effect of Kynurenic Acid On Serum Adenosine Deaminase Activity (ADA) in Hyperuricemic Mice

Serum samples of Example 2 were taken, and ADA enzyme activity levels in the mouse serum samples were determined by using an adenosine deaminase activity (ADA) assay kit (a peroxidase method).

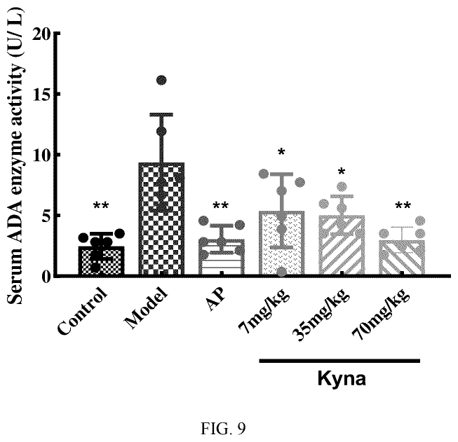

As shown in FIG. 9, the serum ADA enzyme activity levels in the mice of the model group were significantly increased (p<0.01) compared with the blank control group and the positive control group. After administration of KA to the mice, the serum ADA enzyme activity levels in the mice were significantly reduced, reaching a level comparable to those of the blank control group and the positive control group (p<0.01), and the inhibitory effect of KA-H on the serum ADA enzyme activity level in the mice was the most significant. It was indicated that KA could effectively reduce serum ADA enzyme activity in the hyperuricemic mice, thereby limiting the production of uric acid precursors.

### Example 7: Inhibitory Effect of Kynurenic Acid and Structural Analogs Thereof on XOD Enzyme Activity in Vitro

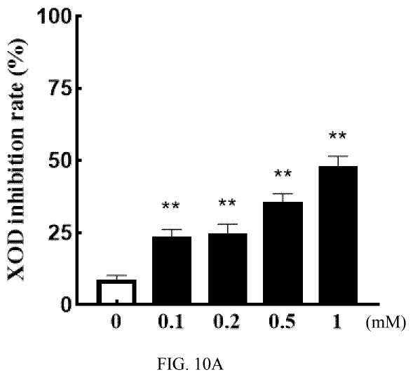

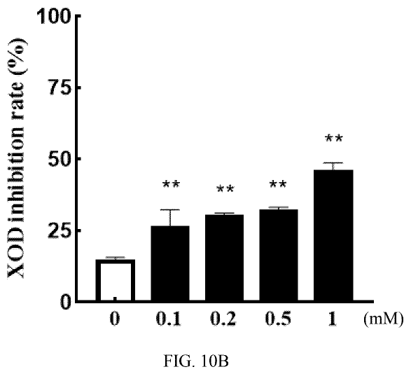

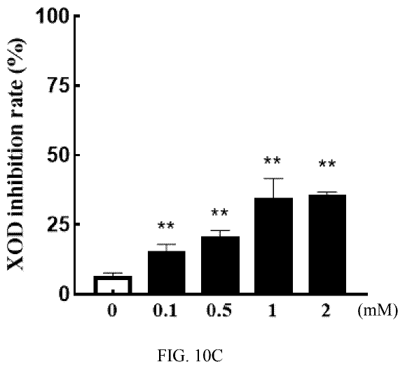

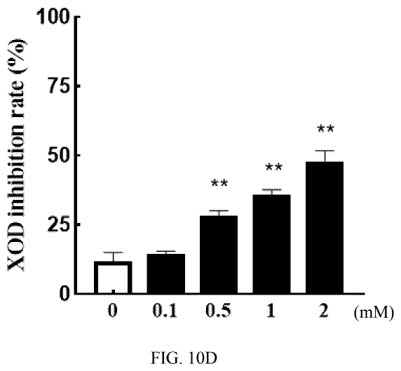

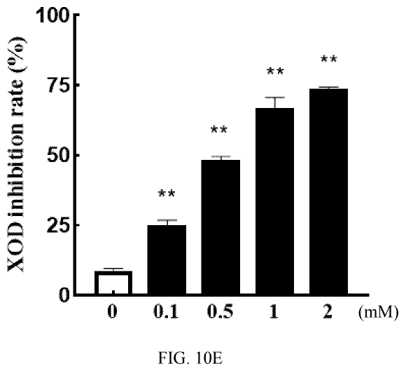

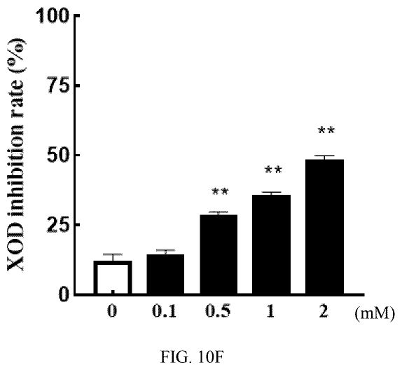

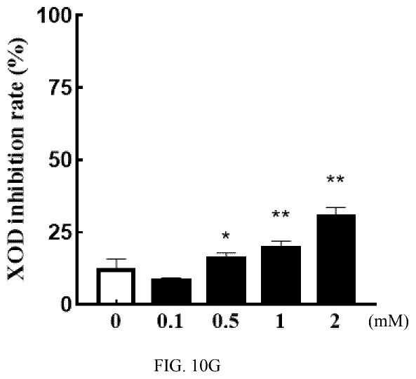

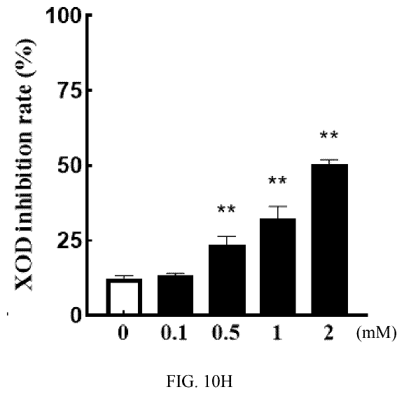

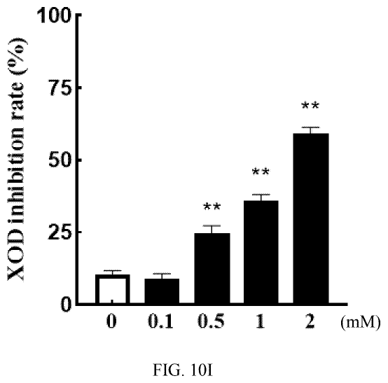

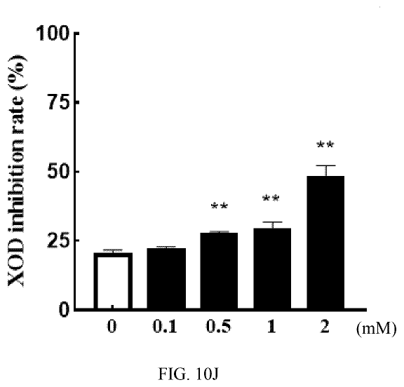

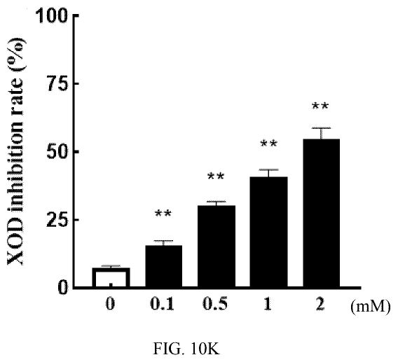

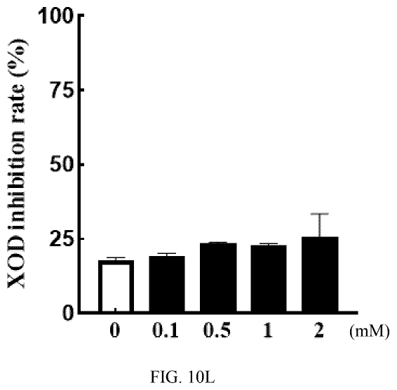

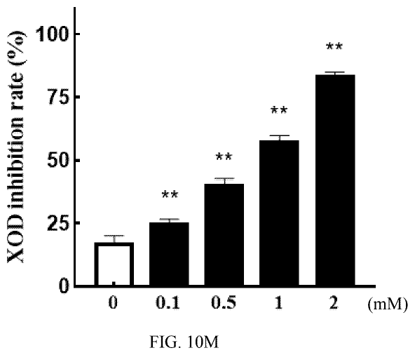

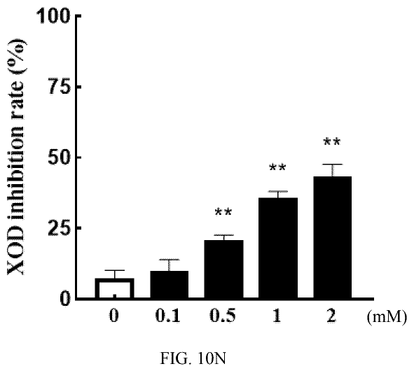

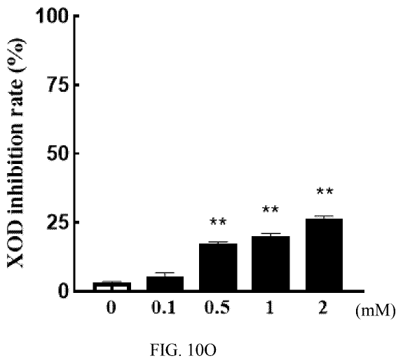

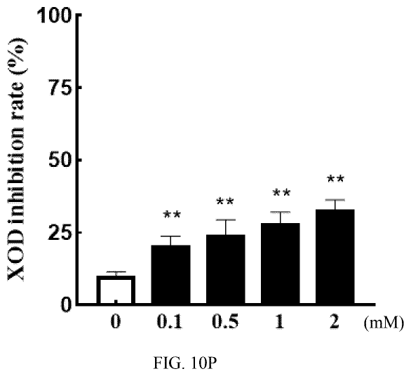

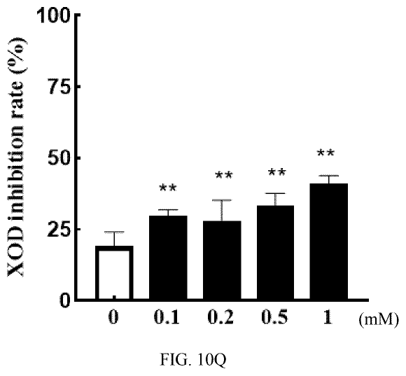

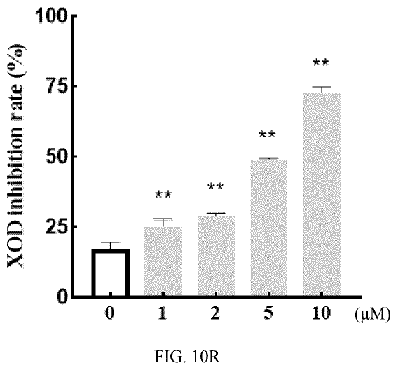

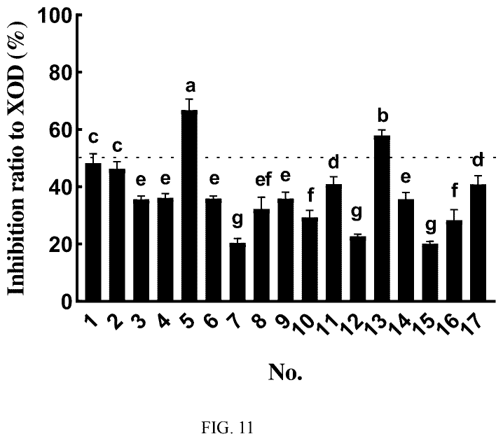

The inhibitory effect of quinoline structural analogs on XOD was evaluated by co-reacting an inhibitor with XOD for 10 min, adding a certain concentration of a reaction substrate xanthine, and measuring the amount of uric acid produced at 290 nm. The inhibitory activity of the quinoline structural analogs on XOD with allopurinol as a positive control is shown in FIG. 10A to 10R. The xanthine oxidase inhibitory activity of 17 targets tested all increased with the increase of concentration. The xanthine oxidase inhibitory activity of the positive control allopurinol was also concentration-dependent in this system, with an IC₅₀ of 4.73±0.52 μM. Compared with other compounds, compounds 5, 7, 11, 13, and KA had better inhibitory effects on XOD, with IC₅₀ (mM) of 0.50±0.13, 5.65±0.10, 1.61±0.07, 0.97±0.05, and 1.99±0.26, respectively (Table 1). Wherein the XOD inhibitory effect of the compound 5 was significantly higher than those of other compounds (p<0.05). In addition, the effect of the structures on the XOD inhibitory activity was analyzed by comparing the xanthine oxidase inhibitory activity of the compounds at the same concentration. As shown in FIG. 11, the xanthine oxidase inhibitory activity of compounds 5 and 13 was more than 50% at a compound concentration of 1 mM. The xanthine oxidase inhibitory activity of the two compounds at this concentration was significantly higher than those of the other compounds (p<0.05).

TABLE 1
XOD inhibitory activity (XOI) of kynurenic acid and structural analogs
thereof

| No. | Name | Structure | MW | XOI_{IC50} |
|---|---|---|---|---|
| 1 | Ethyl 4-hydroxy-6- methoxyquinoline-3-carboxylate | 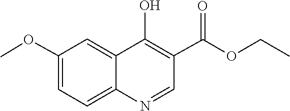 [SMILES: CCOC(=O)c1cnc2ccc(OC)cc2c1O] | 247.25 | |
| 2 | Ethyl 4-hydroxy-8- methoxyquinoline-3-carboxylate | 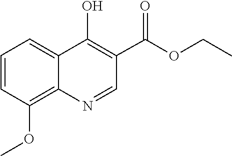 [SMILES: CCOC(=O)c1cnc2c(OC)cccc2c1O] | 247.25 | |
| 3 | Ethyl 4-hydroxyquinoline-3- carboxylate | 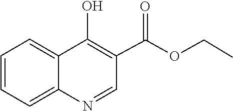 [SMILES: CCOC(=O)c1cnc2ccccc2c1O] | 217.22 | |
| 4 | 4-Hydroxyquinoline-3-carboxylic acid | 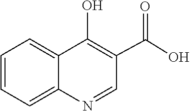 [SMILES: O=C(O)c1cnc2ccccc2c1O] | 189.17 | |
| 5 | 6-Hydroxyquinoline-3-carboxylic acid | 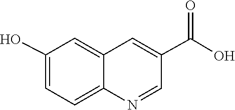 [SMILES: O=C(O)c1cnc2ccc(O)cc2c1] | 189.17 | 0.50 ± 0.13^{E} |
| 6 | 4-Hydroxyquinoline | 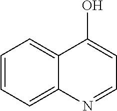 [SMILES: Oc1ccnc2ccccc12] | 145.16 | |
| 7 | Ethyl 4-hydroxyquinoline-2- carboxylate | 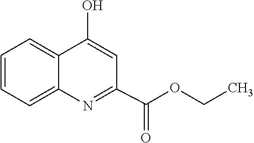 [SMILES: CCOC(=O)c1cc(O)c2ccccc2n1] | 217.220 | 5.65 ± 0.10^{A} |
| 8 | Xanthurenic acid | 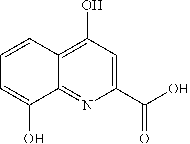 [SMILES: O=C(O)c1cc(O)c2cccc(O)c2n1] | 205.17 | |
| 9 | Kynurenic acid | 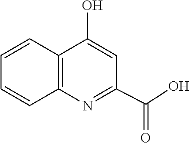 [SMILES: O=C(O)c1cc(O)c2ccccc2n1] | 189.17 | 1.99 ± 0.26^{B} |
| 10 | 5-Hydroxyquinoline-2-carboxylic acid | 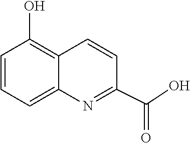 [SMILES: O=C(O)c1ccc2c(O)cccc2n1] | 189.17 | |
| 11 | 2-Hydroxyquinoline-4-carboxylic acid | 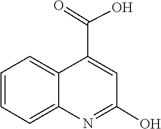 [SMILES: O=C(O)c1cc(O)nc2ccccc12] | 189.17 | 1.61 ± 0.07^{C} |
| 12 | Quinoline-2-carboxylic acid | 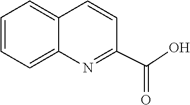 [SMILES: O=C(O)c1ccc2ccccc2n1] | 173.17 | |
| 13 | 3-Hydroxyquinoline-2-carboxylic acid | 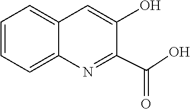 [SMILES: O=C(O)c1nc2ccccc2cc1O] | 189.17 | 0.97 ± 0.05^{D} |
| 14 | 8-Hydroxyquinoline-2-carboxylic acid | 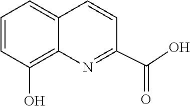 [SMILES: O=C(O)c1ccc2cccc(O)c2n1] | 189.17 | |
| 15 | 8-Hydroxyquinolin-6-carboxylic acid | 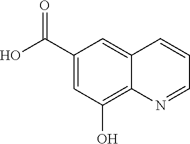 [SMILES: O=C(O)c1cc(O)c2ncccc2c1] | 189.17 | |
| 16 | 8-Hydroxyquinoline-7-carboxylic acid | 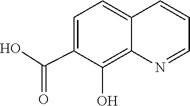 [SMILES: O=C(O)c1ccc2cccnc2c1O] | 189.17 | |
| 17 | 4-Hydroxyquinoline-7-carboxylic acid | 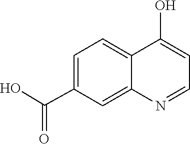 [SMILES: O=C(O)c1ccc2c(O)ccnc2c1] | 189.17 | |

Note: XOI (xanthine oxidase inhibition) activity of allopurinol: IC₅₀: 4.73 ± 0.52 (μM). Capital letters indicate significant differences in the XOD inhibitory activity of compounds (p < 0.05).

In the description in this specification, descriptions with reference to the terms “one example”, “some examples”, “instances”, “specific instances”, or “some instances” or the like mean that particular features, structures, materials, or characteristics described in combination with the examples or instances are included in at least one example or instance of the present disclosure. In this specification, schematic representations of the above terms should not be understood as necessarily referring to the same example or instance. Furthermore, the particular features, structures, materials, or characteristics described may be combined in any suitable manner in one or more examples or instances. Furthermore, different examples or instances described in this specification may be joined and combined by those skilled in the art.

10 Although the examples of the present disclosure have been shown and described above, it will be appreciated that the above examples are illustrative and not to be construed as limiting the present disclosure, and that one of ordinary skill in the art may make changes, modifications, substitutions, and variations to the above examples within the scope of the present disclosure.

What is claimed is:

1. A medicament for ameliorating or treating hyperuricemia, comprising kynurenic acid and a pharmaceutically acceptable carrier, wherein the kynurenic acid has the following structural formula:

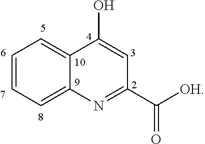 [SMILES: O=C(O)c1cc(O)c2ccccc2n1]

2. A medicament for ameliorating or treating hyperuricemia, comprising a structural analog of kynurenic acid and a pharmaceutically acceptable carrier, wherein the structural analog of the kynurenic acid has the following general structural formula: 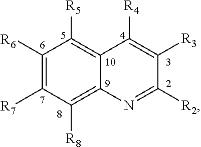
wherein R₃ is ethyl carboxylate, R₄ is hydroxy, and R₆ is methoxy; or R₃ is carboxyethyl ester, R₄ is hydroxy, and R₈ is methoxy; or R₃ is ethyl carboxylate, and R₄ is hydroxy; or R₃ is carboxylic acid and R₄ is hydroxy; or R₃ is carboxylic acid, and R₆ is hydroxy; or R₄ is hydroxy; or R₂ is ethyl carboxylate, and R₄ is hydroxy; or R₂ is carboxylic acid, R₄ is hydroxy, and R₈ is hydroxy; or R₂ is carboxylic acid, and R₅ is hydroxy; or R₂ is hydroxy, and R₄ is carboxylic acid; or R₂ is carboxylic acid; or R₂ is carboxylic acid, and R₃ is hydroxy; or R₂ is carboxylic acid, and R₈ is hydroxy; or R₆ is carboxylic acid and R₈ is hydroxy; or R₇ is carboxylic acid and R₈ is hydroxy; or R₄ is hydroxy and R₇ is carboxylic acid.

3. The medicament for ameliorating or treating hyperuricemia according to claim 2, wherein the structural analog of the kynurenic acid is ethyl 4-hydroxy-6-methoxyquinoline-3-carboxylate, ethyl 4-hydroxy-8-methoxyquinoline-3-carboxylate, ethyl 4-hydroxyquinoline-3-carboxylate, 4-hydroxyquinoline-3-carboxylic acid, 6-hydroxyquinoline-3-carboxylic acid, 4-hydroxyquinoline, ethyl 4-hydroxyquinoline-2-carboxylate, xanthurenic acid, 5-hydroxyquinoline-2-carboxylic acid, 2-hydroxyquinoline-4-carboxylic acid, quinoline-2-carboxylic acid, 3-hydroxyquinoline-2-carboxylic acid, 8-hydroxyquinoline-2-carboxylic acid, 8-hydroxyquinoline-6-carboxylic acid, 8-hydroxyquinoline-7-carboxylic acid, or 4-hydroxyquinoline-7-carboxylic acid.
